(19) United States
(12) Patent Application Publication
(10) Pub. No.: US 2014/0251005 A1
(43) Pub. Date: Sep. 11, 2014

(54) Connected Tableware for Quantifying Dietary Intake

(71) Applicant: Beam Technologies, LLC, Louisville, KY (US)

(72) Inventors: Alexander D. Curry, Louisville, KY (US); Alex X. Frommeyer, Louisville, KY (US); Daniel E. Dykes, Louisville, KY (US)

(73) Assignee: Beam Technologies, LLC, Louisville, KY (US)

(21) Appl. No.: 14/204,586

(22) Filed: Mar. 11, 2014

Related U.S. Application Data
(60) Provisional application No. 61/775,815, filed on Mar. 11, 2013.

(51) Int. Cl. G01N 33/02; G01K 13/00; G01N 27/04; G01G 19/414; G01F 23/00; G01N 21/47

(52) U.S. Cl. CPC G01N 33/02; G01F 23/0007; G01N 21/47; G01N 27/04; G01G 19/4146; G01K 13/00
USPC 73/290 R; 73/866; 250/338.1; 324/693; 177/2; 374/141

(57) ABSTRACT

Connected tableware is provided with various sensors and systems for quantifying a user's dietary intake. Sensors and systems include a weight sensing system, a fluid level sensor, a fat content sensor, and a temperature sensor. Further, the connected tableware is comprised in a system including a data transfer medium (i.e. “smartphone”) and the Cloud, which allows for data transfer between multiple platforms from the connected tableware. The connected tableware and the system operate in conjunction to identify the dietary items contained on the connected tableware, thus quantifying dietary intake of a user.

### CROSS-REFERENCE TO RELATED APPLICATIONS

This application claims priority to U.S. Provisional Application No. 61/775,815 filed on Mar. 11, 2013.

### BACKGROUND OF THE INVENTION

This invention relates to quantifying dietary intake, particularly relating to connected tableware and systems for quantifying dietary intake. In particular, the invention relates to the measurement of food quantities placed on tableware and thereby determining the dietary intake of the user.

A common task for health conscious people is to quantify and track their dietary intake in the form of calories and other relevant nutritional facts. The current methods for quantifying calories are sufficient in the method of correlating a certain amount of something to a specific number calories but severely lack in the method for obtaining the specific amount of food or beverage that is being eaten or drank. Current methods that exist include the use of food scales and eating premeasured packaged foods. Current methods are limiting in the type of foods that can be eaten and where those foods can be eaten.

### BRIEF SUMMARY OF THE INVENTION

The invention aims to provide connected tableware for quantifying dietary intake of a user. The connected tableware is comprised in the form of various tableware items (i.e. plate, cup, bowl, bottle, etc.) that comprise added electronics capability. The added electronics capability of connected tableware comprises a data processing unit for storing and processing data and a transceiver for transmitting and receiving data.

Food or beverage is placed in the respective connected tableware item, wherein the amount of said food or beverage is measured. The user then identifies the food or beverage present and the nutritional content of the food or beverage is calculated. Additionally, if the meal is completed with food or beverage remaining, the user can perform the measurement and identification steps again to determine a more accurate nutritional intake.

Accordingly several advantages are to provide connected tableware, to provide a means for quantifying dietary intake, to provide an improved method for determining caloric intake, and to provide a means for easily delivering food quantities to common devices used for tracking caloric intake. Still further advantages will become apparent from a study of the following descriptions and the accompanying drawings.

### BRIEF DESCRIPTION OF THE DRAWINGS

FIG. 1 is a schematic view of the components of a connected tableware apparatus with a measurement sensor according to multiple embodiments and alternatives.

FIG. 2 is a schematic view of the components of a connected tableware apparatus with a weight sensing system according to multiple embodiments and alternatives.

FIG. 3 is a perspective view of a connected tableware apparatus embodied as a dish according to multiple embodiments and alternatives.

FIG. 4 is a perspective view of a connected tableware apparatus with a segmented configuration according to multiple embodiments and alternatives.

FIG. 5 is a schematic view of the components of a connected tableware apparatus with a fluid level sensor according to multiple embodiments and alternatives.

FIG. 6 is a perspective view of a connected tableware apparatus embodied as a cup according to multiple embodiments and alternatives.

FIG. 7 is a schematic view of the components of a connected tableware apparatus with a fat content sensor according to multiple embodiments and alternatives.

FIG. 8 is a schematic view of the components of a connected tableware apparatus with a temperature sensor according to multiple embodiments and alternatives.

FIG. 9 is a schematic view of a connected tableware system according to multiple embodiments and alternatives.

### DETAILED DESCRIPTION OF THE INVENTION

The connected tableware for quantifying dietary intake is encompassed in a plurality of embodiments that shall be discussed in the present section.

A plurality of embodiments comprises tableware, wherein tableware includes dishes, bowls, cups and bottles. Tableware is used for holding food and beverages, broadly dietary intake, for consumption by a user. Tableware provides the user with the opportunity to quantify his/her dietary intake prior to consumption. Thus, tableware provides the user with the optimum interface to quantify dietary intake prior to consumption without changing dietary sequences, as is required with the use of a food scale. Inherently, common tableware does not contain the capability to capture and process data about the dietary intake it holds. Therefore, the integration of technological advancements is required to provide the capability to capture and process data.

Consequently, the tableware of the present invention comprises a data processing unit 127 having at least one collector, a storage medium, and at least one processor, wherein the collector, storage medium, and processor, respectively, collect, store, and process data. Accordingly, the data processing unit 127 is chosen from the group microprocessor, microcontroller, field programmable gate array (FPGA), digital signal processing unit (DSP), application specific integrated circuit (ASIC), programmable logic, and combinations thereof.

Additionally, in some embodiments, the collector of the data processing unit 127 is an electrically conductive wire, wherein the electrically conductive wire receives the electrical output of various sensors.

Moreover, the storage medium of the data processing unit 127 is comprised of volatile memory and non-volatile memory, wherein volatile memory is used for short-term storage and processing, and non-volatile memory is used for long-term storage. Accordingly, volatile memory is chosen from the group random-access memory (RAM), dynamic random-access memory (DRAM), double data rate synchronous dynamic random-access memory (DDR SDRAM), static random-access memory (SRAM), thyristor random-access memory (T-RAM), zero-capacitor random-access memory (Z-RAM), and twin transistor random-access memory (TTRAM). Non-volatile memory is chosen from the group read-only memory (ROM), programmable read-only memory (PROM), erasable programmable read-only memory (EPROM), electrically erasable programmable read-only memory (EEPROM), flash memory, ferroelectric random-access memory (FeRAM), magnetoresistive random-access memory (MRAM), phase-change memory (PRAM), conductive-bridging random-access memory (CBRAM), silicon-oxide-nitride-oxide-silicon memory (SONOS), resistive random-access memory (RRAM), racetrack memory, nano-random-access memory (NRAM), and Millipede memory.

The processor of the data processing unit 127 is chosen from the group microprocessor and micro controller.

Additionally, the connected tableware for quantifying dietary intake comprises at least one data transmitter 134, such that the data can be transmitted to be used by another medium and data can be received from another medium. The data is packaged as at least one signal and transmitted to another medium. The data transmitter 134 is chosen form the group universal serial bus (USB), serial port, wired Ethernet port, radio frequency, microwave communication, infrared short-range communication, near field communication, and Bluetooth®.

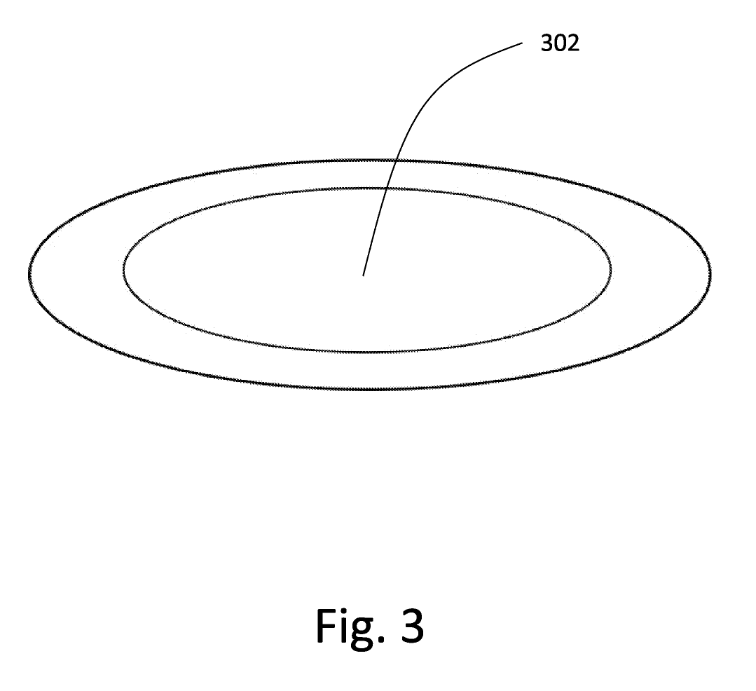

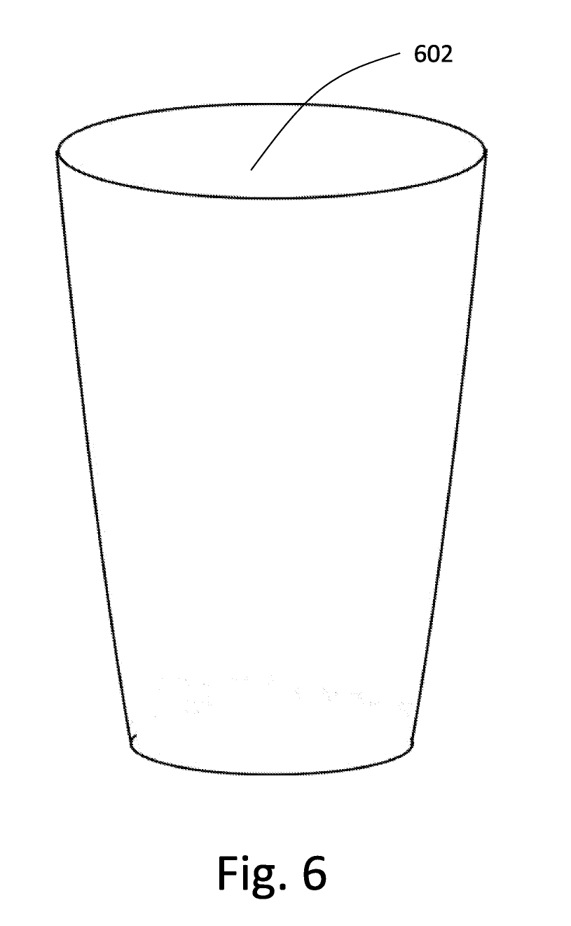

FIG. 3 shows an embodiment of the connected tableware having a holding surface 302. The holding surface 302 is configured to at least partially support dietary items, such as food or beverage. Similarly, FIG. 6 shows an embodiment of the connected tableware having a holding surface 602.

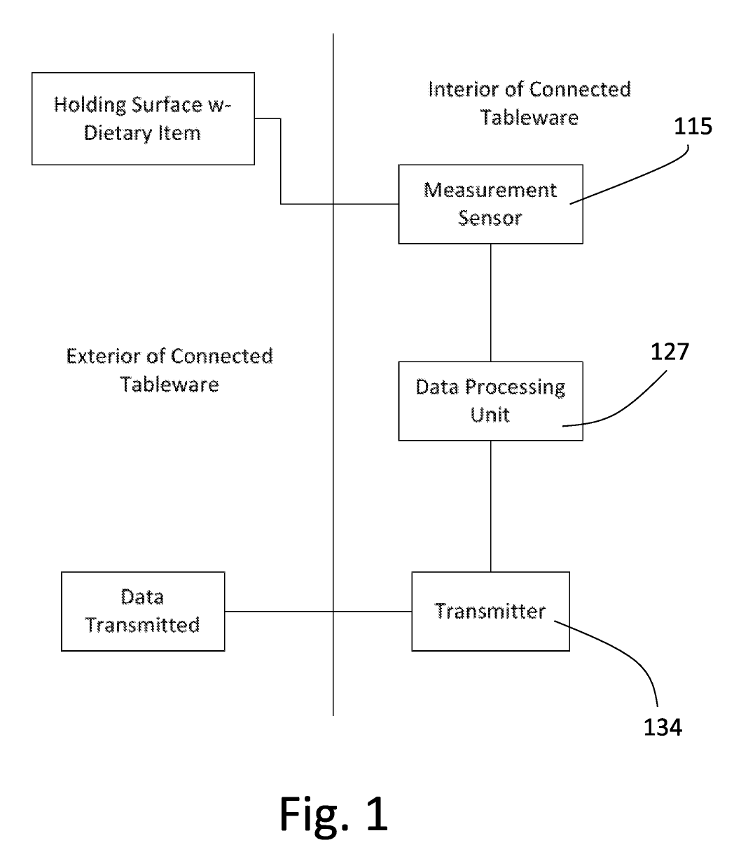

Referring now to FIG. 1, the connected tableware comprises a measurement sensor 115 for measuring at least one characteristic of at least one dietary item placed on the holding surface of the connected tableware. The measurement sensor 115 is comprised in a plurality of embodiments that provide for the measurement of various characteristics of dietary items. Embodiments of the measurement sensor shall be further discussed in this section.

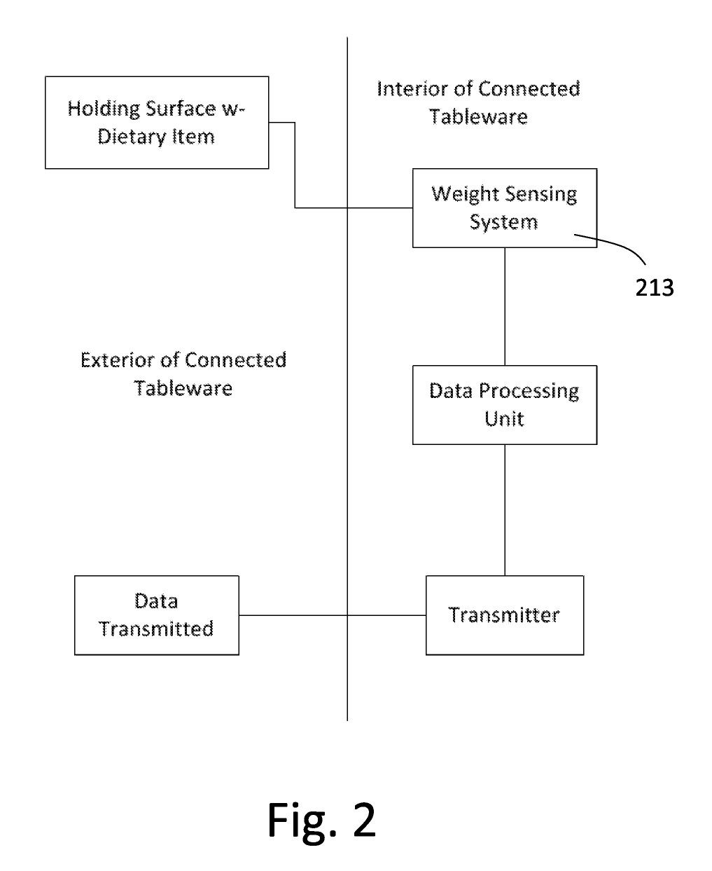

As shown in FIG. 2, the connected tableware comprises a weight sensing system 213 to collect weight measurements of dietary intake. The weight sensing system 213 is comprised of either a segmented configuration, open configuration, or any combination thereof.

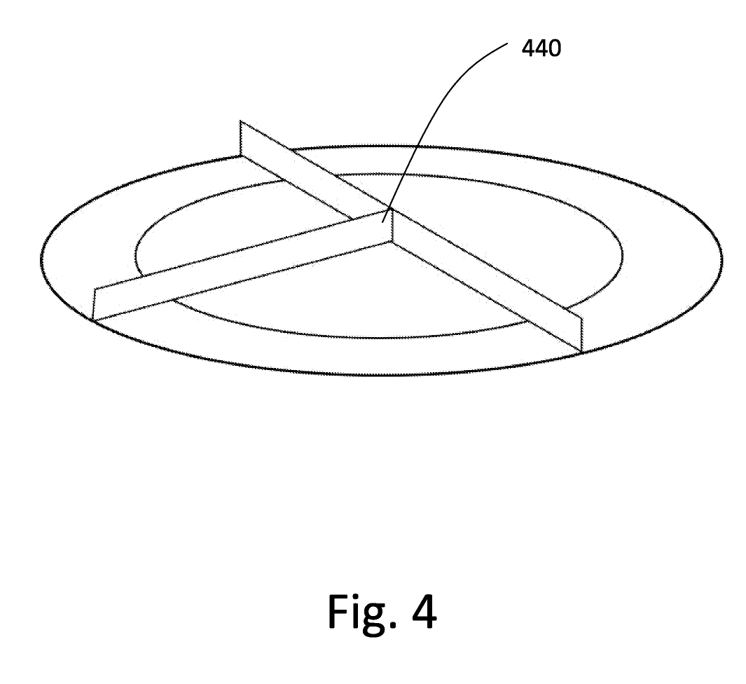

Referring now to FIG. 4, a segmented configuration 440 of the weight sensing system comprises a physical or immaterial barrier that separates the whole of the device's holding capacity into smaller distinct or definite segments that allow the user to identify individual segments. The individual segments provide for easy separation of different substances, such as dividing meat and vegetables. Consequently in this configuration, the user places separate substances in individual segments for easy identification of the substance being weighed, thus providing more accurate quantities of dietary intake.

Often, the segmented configuration of the weight sensing system comprises a plurality of weight sensors, commonly referred to as scales, wherein at least one weight sensor is comprised within each segment of the device. Accordingly, at least one weight sensor is utilized to measure the weight of a certain segment that contains a certain substance, which allows the user to calculate the caloric content of the item based on its weight and existing data that correlates caloric content to weight. The segmented configuration enables the user to easily portion an entire meal within the segments of the device while quickly and accurately obtaining the weight of each item that will be consumed, thus allowing for the calculation of caloric content for the user's dietary intake.

The weight sensor of the segmented configuration of the weight sensing system is chosen from the group strain gage, electronic analytical scale, capacitive scale, and any combination thereof. Commonly, a strain gage is comprised of an insulating flexible backing with a metallic foil pattern affixed and is attached to the object to be measured in a proper place with an adhesive, such as cyanoacrylate. As a load is applied to the object, the object deforms causing the foil of the strain gage to deform that results in an electrical resistance change, which is typically measured using a Wheatstone bridge. The resistance change is converted into a strain value using a gage factor and the strain value is used to calculate the load on the object, which provides the weight of the item providing load on the object.

An electronic analytical scale measures the load on an object by countering the load applied using an electromagnet to generate a force. The measurement of the counter force applied by the electromagnet and the electromagnet itself are often comprised in an electromagnetic force restoration sensor.

A capacitive scale comprises a capacitive sensor that is constructed from two parallel conductive plates separated by an insulator such that, in the active portion of the sensor, the insulator allows for an air gap between the parallel plates. Forces acting perpendicular to the plane of the parallel plates in the active region deform one or both conductors. Accordingly, the parallel plates move closer together or further apart due to deformation, thus, changing the capacitance of the sensor. The change in capacitance is scaled by a reference factor that provides the weight of the item causing the capacitance change.

An open configuration of the weight sensing system resembles a normal tableware item such that the holding surface of the item is indistinguishable from a typical tableware item, essentially lacking barriers. The user places dietary items on the plate as normal without regard for segmentation. A weight sensing system having an open configuration comprises a network of weight sensors such that the weight of dietary items dispersed across the holding surface can be measured.

Optionally, a weight sensing system having an open configuration is comprised of a network of strain gages, wherein strain gages are optimally placed throughout the underside of the holding surface and each strain gage has an associated position relative to a reference point. The dispersed dietary items create concentrated loads along the holding surface of the connected tableware. The network of strain gages operates in conjunction with the data processing unit to identify the position of dietary items that create concentrated loads and measures the weight of the concentrated load on the holding surface. Thus identifying individual weighing individual dietary items dispersed throughout the holding surface.

In further options, a weight sensing system having an open configuration is comprised of a network of capacitive scales and operates under the same principles as the previously described network of strain gages to determine the position and weight of concentrated loads.

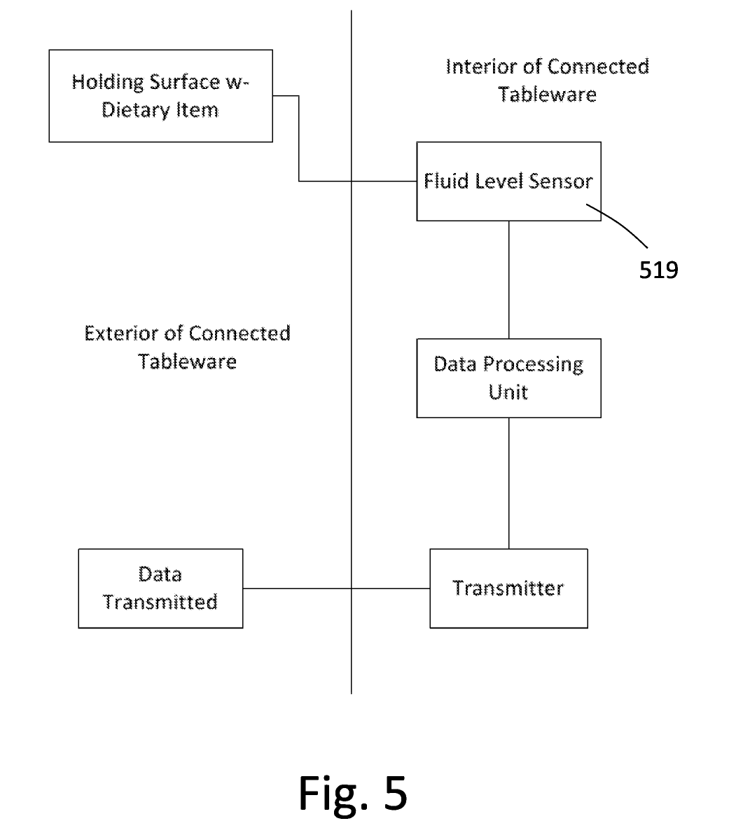

Referring to FIG. 5, the connected tableware further comprises a fluid level sensor 519 that detects the level of the dietary fluid held by the connected tableware. Additionally, the fluid level sensor 519 may be configured to signal when the dietary fluid level has achieved a specified or predefined level. The fluid level sensor 519 is chosen from the group displacement level gage, strain gage, capacitance transmitter, magnetostrictive level transmitter, ultrasonic level transmitter, radar level transmitter, and any combination thereof.

A displacement level gauge comprises a column of solid material (a displacer) that is suspended in the holding area of the tableware. As the fluid volume in the holding area increases, a buoyant force pushes up on the displacer. The displacer is calibrated to be displaced when the fluid volume changes. As the displacer is displaced, it changes the force exerted on a force transducer that has consistent contact with the displacer. The changes in the force exerted by the displacer correspond to changes in dietary fluid level.

Optionally, at least one strain gage is affixed to the underside of the holding area such that distortions in the holding area are quantitatively measured. As the fluid volume increases, the force applied to and thus distortion exhibited by the holding area increases. The changes in the force measured by the strain gage correspond to changes in dietary fluid level.

A capacitance transmitter comprises either an insulated rod connected to a transmitter and the fluid, or an uninsulated rod attached to a transmitter and the reservoir wall. As the volume of fluid changes, the overall capacitance introduced by the rod changes proportionately. A capacitance bridge measures the capacitance of the rod and is calibrated to correlate specified capacitances to dietary fluid levels.

A magnetostrictive level transmitter comprises a sensor wire, piezoceramic sensor, transmitter, and magnetic float. The sensor wire runs through the center of the magnetic float and is attached to the piezoceramic sensor. The fluid level is detected by the transmitter sending a short current pulse down the sensor wire, which sets up a magnetic field along the length of the sensor wire. A timing circuit is also triggered on when the pulse is sent. The magnetic float reacts to the magnetic field and produces a torsional force in the sensor wire. The torsional force is detected by the piezoceramic sensor which sends out a varying electric signal dependent on the fluid level.

An ultrasonic level transmitter comprises a transducer and timing circuit. The transducer transmits ultrasound to the surface of the fluid. The timing circuit measures the amount of time it takes the ultrasound wave to travel back to the transducer. The amount of time corresponds to fluid level of the holding area. A radar level transmitter operates in the same manner as the ultrasonic level transmitter with exception of transmitting microwaves, which are reflected from the fluid surface.

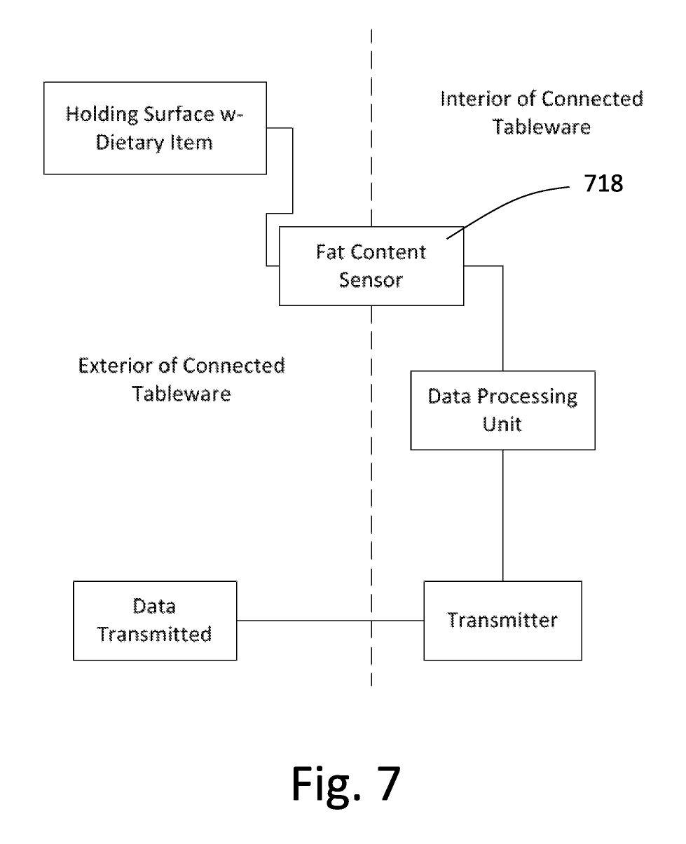

Referring now to FIG. 7, the connected tableware further comprises a fat content sensor 718 that measures the fat content of certain dietary items prior to consumption. The fat content sensor 718 is integrated into the holding surface of the connected tableware such that fat content is measured when the dietary items are placed on the holding surface of the connected tableware. The fat content measurement provides further nutritional facts of the dietary intake of the user, which allows the user to not only track caloric intake but also fat intake.

Optionally, the fat content sensor 718 utilizes the method of near-infrared interactance comprising at least one infrared emitter and at least one photo detector. A beam of infrared light is transmitted into the dietary item. The infrared light is reflected by non-fatty matter and absorbed by the fatty matter. The photo detector captures the reflected infrared light. The ratio of the infrared light returned to the photo detector to the infrared light emitted by the infrared emitter is correlated to the percentage of fat content in the dietary item.

Further variations of the fat content sensor 718 include electrical impedance analysis comprising at least two conductors. A small electric current is sent through the dietary item and the resistance between the conductors is measured. The resistance is correlated to fat content percentage of the dietary item. In general, fat is anhydrous and a poor conductor of electric current. Conversely, non-fatty matter, water, and electrolytes are good conductors of electric current.

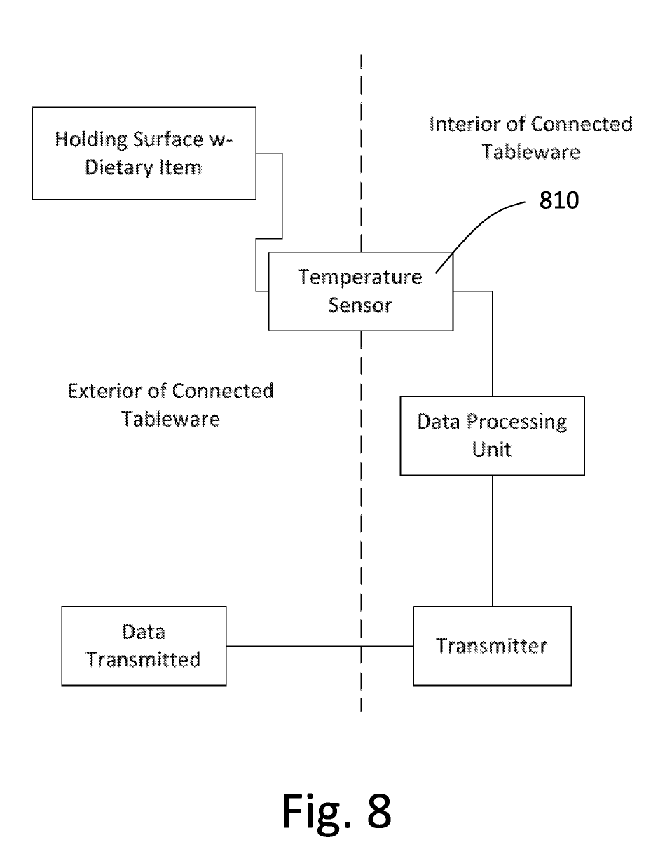

As shown in FIG. 8, the connected tableware further comprises at least one temperature sensor 810 that measures the temperature of dietary items prior to and during consumption. When dietary items are placed on the holding surface of the tableware, at least one temperature sensor 810 collects the temperature of the various dietary items placed on the holding surface. This allows the user to determine if dietary items are prepared to a proper and/or safe temperature, such as chicken or pork where illnesses can result from poor preparation.

In some embodiments, the temperature sensor 810 is at least one thermocouple, wherein the thermocouple comprises two different conductors, typically metal alloys, that produce a voltage proportional to a temperature difference between either end of the pair of conductors. Optionally, the temperature sensor 810 is at least one thermistor, wherein the thermistor is a resistor that has a certain resistance, which varies significantly with temperature. Thermistors are generally comprised of a ceramic or polymer material.

Optionally, the temperature sensor 810 is at least one resistance temperature detector (RTD), wherein the RTD exploits a predictable change in electrical resistance that is dependent upon a change in temperature. Often, the material of the RTD is platinum. Alternatively, the temperature sensor 810 is at least one infrared temperature sensor, wherein the temperature of an object is determined by a portion of thermal radiation referred to as blackbody radiation emitted by the object, such that knowing the infrared energy emitted and the object's emissivity allows for the determination of the object's temperature.

Optionally, the temperature sensor 810 is at least one thermopile, wherein the thermopile converts thermal energy into electrical energy and is comprised of one or more thermocouples connected in series or parallel. Optionally, the temperature sensor 810 is at least one thermostat, wherein the thermostat comprises two different metals that are bonded together to form a bi-metallic strip, such that the difference in linear expansion rates causes a mechanical bending movement when heat is applied. In some embodiments, the temperature sensor 810 is at least one silicon bandgap temperature sensor, wherein the forward voltage of a silicon diode is dependent on temperature, and the temperature is determined by comparing bandgap voltages at two different currents.

Optionally, the temperature sensor 810 further comprises at least one protrusion that extends from the holding surface of the connected tableware such that the protrusion can be inserted into dietary items to obtain said dietary item's internal temperature. Often, the protrusion is an extension of the temperature sensor such that the temperature sensor 810 is located at any point in contact with the protrusion including the distal tip of the protrusion. Further, the protrusion can be retractable, wherein it extends beyond the holding surface of the connected tableware only when a temperature measurement is being taken.

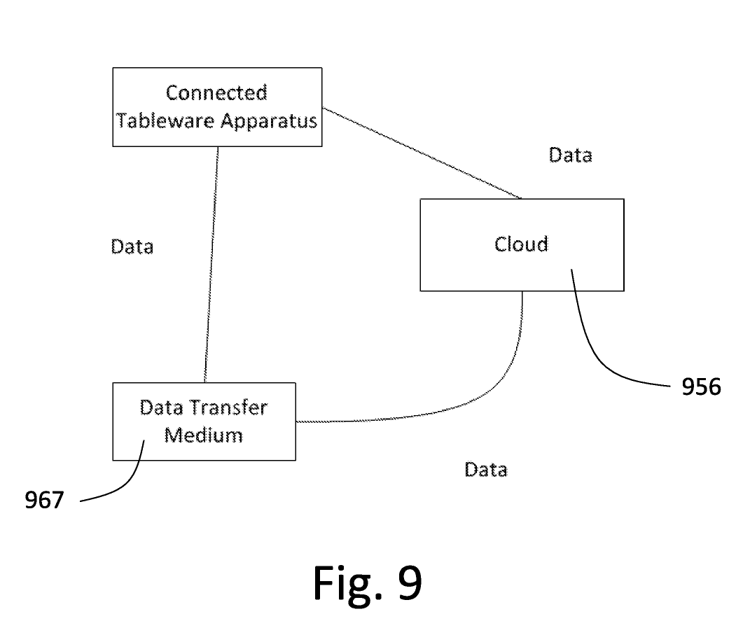

Referring now to FIG. 9, the connected tableware for quantifying dietary intake including variations described herein is comprised in a system that allows a user to view and monitor the measured data via a data transfer medium 967, such as a “smartphone”, and/or a network storage device, often known as the “cloud” and hereinafter referred to as the Cloud 956. Embodiments of the connected tableware comprised in this system include the data transmitter described previously. Accordingly, the system allows the connected tableware to transfer data to the data transfer medium 967 and/or the Cloud 956. Additionally, the data transfer medium 967 may transfer said data to the Cloud 956 for display and manipulation on further data transfer mediums connected to said Cloud 956. Alternatively, the Cloud 956 may transfer said data to the data transfer medium 967.

In some embodiments, the data transfer medium 967 comprises a receiver, a transmitter, a data processing unit, and a display. Accordingly, the data processing unit is chosen from the group microprocessor, microcontroller, field programmable gate array (FPGA), digital signal processing unit (DSP), application specific integrated circuit (ASIC), programmable logic, and combinations thereof. The data processing unit comprises a collector, storage medium, and a processor.

Moreover, the storage medium of the data processing unit is comprised of volatile memory and non-volatile memory, wherein volatile memory is used for short-term storage and processing, and non-volatile memory is used for long-term storage. Accordingly, in some embodiments, volatile memory is chosen from the group random-access memory (RAM), dynamic random-access memory (DRAM), double data rate synchronous dynamic random-access memory (DDR SDRAM), static random-access memory (SRAM), thyristor random-access memory (T-RAM), zero-capacitor random-access memory (Z-RAM), and twin transistor random-access memory (TTRAM). Optionally, in some embodiments, non-volatile memory is chosen from the group read-only memory (ROM), programmable read-only memory (PROM), erasable programmable read-only memory (EPROM), electrically erasable programmable read-only memory (EEPROM), flash memory, ferroelectric random-access memory (FeRAM), magnetoresistive random-access memory (MRAM), phase-change memory (PRAM), conductive-bridging random-access memory (CBRAM), silicon-oxide-nitride-oxide-silicon memory (SONOS), resistive random-access memory (RRAM), racetrack memory, nano-random-access memory (NRAM), and Millipede memory.

Further still, the processor of the data processing unit is chosen from the group microprocessor and microcontroller.

Additionally, the receiver of the data transfer medium 967 is chosen from the group universal serial bus (USB), serial port, wired Ethernet port, radio frequency, microwave communication, infrared short-range communication, near field communication, and Bluetooth. Often, the receiver of the data transfer medium 967 receives at least one signal from the data extractor of the toothbrush.

Optionally, the data transfer medium 967 is chosen from the group personal computer, tablet computer, mobile phone (i.e. “smartphone”), television, dedicated system, charging station, network router, and web-enabled server.

Optionally, the transmitter of the data transfer medium 967 is chosen from the group universal serial bus (USB), serial port, wired Ethernet port, radio frequency, microwave communication, infrared short-range communication, near field communication, and Bluetooth.

Additionally, the display of the data transfer medium 967 converts signals into user-readable formats.

In some embodiments, the Cloud 956 is connected to a network, wherein the network is chosen from the group Internet or intranet such that an intranet is a network managed and accessed by an internal organization and is not accessible to the outside world. The network is utilized by the Cloud 956 for receiving and transmitting data. The mode for receiving and transmitting data through the network is chosen from the group universal serial bus (USB), serial port, wired Ethernet port, radio frequency, microwave communication, infrared short-range communication, near field communication, and Bluetooth.

Additionally, the Cloud 956 processes data using at least one microprocessor, at least one microcontroller, or a combination thereof. The storage of data is comprised of volatile memory and non-volatile memory, wherein volatile memory is used for short-term storage and processing, and non-volatile memory is used for long-term storage. Accordingly, volatile memory is chosen from the group random-access memory (RAM), dynamic random-access memory (DRAM), double data rate synchronous dynamic random-access memory (DDR SDRAM), static random-access memory (SRAM), thyristor random-access memory (T-RAM), zero-capacitor random-access memory (Z-RAM), and twin transistor random-access memory (TTRAM). Optionally, non-volatile memory is chosen from the group read-only memory (ROM), programmable read-only memory (PROM), erasable programmable read-only memory (EPROM), electrically erasable programmable read-only memory (EEPROM), flash memory, ferroelectric random-access memory (FeRAM), magnetoresistive random-access memory (MRAM), phase-change memory (PRAM), conductive-bridging random-access memory (CBRAM), silicon-oxide-nitride-oxide-silicon memory (SONOS), resistive random-access memory (RRAM), racetrack memory, nano-random-access memory (NRAM), and Millipede memory.

The Cloud 956, optionally, is a network server primarily used for storing and processing data. Optionally, the Cloud 956 is comprised of more than one network server such that the network servers operate in conjunction to increase the storing and processing capabilities of the Cloud. Alternatively, the Cloud 956 is provided as a service such that it is physically located at a location separate from the user, and the service provided is the storing and processing of data.

Additionally, the system comprising the connected tableware allows the user to specify the identity of dietary items contained on the holding surface of the connected tableware. The connected tableware operates in conjunction with the data transfer medium to identify individual dietary items on the holding surface. Optionally, the connected tableware identifies a portion of the holding surface that holds a dietary item. Concurrently, the data transfer medium prompts the user to input the type of dietary item, thus quantifying the dietary intake of the user. Optionally, the data transfer medium identifies a dietary item on the holding surface and prompts the user to input the type of dietary item.

In some embodiments, the system comprising the connected tableware facilitates the user's participation in social games related to the data collected by the sensors of the connected tableware. Participation in said social games is accomplished passively through the collection of data by the sensors of the connected tableware over a period of time, rather than participation by real-time user input. Optionally, the social games consist of goals to be accomplished, competitive games between multiple users or between a singular user and a computer generated user, and challenges to complete specified milestones.

Participation in social games is accomplished through a plurality of different user groups. The first user group for participation is a closed loop user group, which is accomplished on a specific data transfer medium and participation is limited to the users of said specific data transfer medium. The second user group for participation is a networked user group, which is accomplished over a network that connects a plurality of data transfer mediums. Networked user groups are further defined as including users belonging to a certain group defined through social media or other means. The third user group for participation is a global user group, which is a user group that anyone can join and participate in. The global user group, in some embodiments, may be sponsored or promoted by a particular entity as a form of advertisement or incentive to the users of the global user group.

Participation in social games may be incentivized with an offered reward to encourage participation of members of a user group. Rewards may include coupons, discounts on goods or services, virtual currency, insurance discounts, and customized incentives. Rewards have the advantage of being given based off of passive data collected by sensors, thus rewarding users for health compliance and health statistics.

It will be understood that the embodiments described herein are not limited in their application to the details of the teachings and descriptions set forth, or as illustrated in the accompanying figures. Rather, it will be understood that connected tableware for quantifying dietary intake, as taught and described according to multiple embodiments disclosed herein, is capable of other embodiments and of being practiced or carried out in various ways.

Also, it is to be understood that the phraseology and terminology used herein is for the purpose of description and should not be regarded as limiting. The use herein of “including,” “comprising,” “i.e.,” “containing,” or “having,” and variations of those words is meant to encompass the items listed thereafter, and equivalents of those, as well as additional items.

Accordingly, the descriptions herein are not intended to be exhaustive, nor are they meant to limit the understanding of the embodiments to the precise forms disclosed. It will be understood by those having ordinary skill in the art that modifications and variations of these embodiments are reasonably possible in light of the above teachings and descriptions.

What is claimed is:

1. A connected tableware apparatus, comprising:
a holding surface that is configured to at least partially supports at least one dietary item;
a data processing unit having at least one collector that is configured to collect data, a storage medium that is configured to store data, and at least one processor that is configured to process data;
a transceiver that is configured to transmit data; and
a measurement sensor that is configured to measure at least one characteristic of at least one dietary item.

2. The connected tableware apparatus of claim 1, wherein the transceiver is further configured to receive data.

3. The connected tableware apparatus of claim 1, wherein the connected tableware apparatus is chosen from the group consisting of a dish, bowl, cup, bottle, and any combination thereof.

4. The connected tableware apparatus of claim 1, wherein the transceiver is chosen from the group consisting of a universal serial bus, serial port, wired Ethernet port, radio frequency, microwave communication, infrared short-range communication, near field communication, Bluetooth, Wi-Fi, and any combination thereof.

5. The connected tableware apparatus of claim 1, wherein the measurement sensor is at least one weight sensing system that is configured to measure the weight of at least one dietary item.

6. The connected tableware apparatus of claim 5, wherein at least one weight sensing system comprises at least one weight sensor that is chosen from the group consisting of a strain gage, electronic analytical scale, capacitive scale, and any combination thereof.

7. The connected tableware apparatus of claim 5, wherein the holding surface further comprises a configuration that is chosen from the group consisting of a segmented configuration, open configuration, and any combination thereof.

8. The connected tableware apparatus of claim 7, wherein the segmented configuration further comprises a barrier that is chosen from the group consisting of a physical barrier, immaterial barrier, and any combination thereof.

9. The connected tableware apparatus of claim 7, wherein weight sensing system and the open configuration allow for the measurement and identification of concentrated loads applied by at least one dietary item.

10. The connected tableware apparatus of claim 1, wherein the data processing unit correlates at least one characteristic of at least one dietary item as measured by the measurement sensor to caloric content of said dietary item.

11. The connected tableware apparatus of claim 1, wherein the measurement sensor is at least one fluid level sensor that is configured to detect the fluid level of at least one dietary item.

12. The connected tableware apparatus of claim 11, wherein the fluid level sensor is chosen from the group consisting of a displacement level gage, strain gage, capacitance transmitter, magnetostrictive level transmitter, ultrasonic level transmitter, radar level transmitter, and any combination thereof.

13. The connected tableware apparatus of claim 1, wherein the measurement sensor is at least one fat content sensor that is configured to measure the fat content of at least one dietary item.

14. The connected tableware apparatus of claim 13, wherein the fat content sensor based on a principle chosen from the group consisting of near-infrared interactance, electrical impedance analysis, and any combination thereof.

15. The connected tableware apparatus of claim 1, wherein the measurement sensor is at least one temperature sensor that is configured to measure the temperature of at least one dietary item.

16. The connected tableware apparatus of claim 15, wherein the temperature sensor and the data processing unit operate in conjunction to determine if at least one dietary item is prepared to a proper temperature.

17. The connected tableware apparatus of claim 15, wherein the temperature sensor protrudes from the holding surface and is configured to be inserted into at least one dietary item.

18. A connected tableware apparatus, comprising:
a holding surface that is configured to at least partially supports at least one dietary item;
a data processing unit having at least one collector that is configured to collect data, a storage medium that is configured to store data, and at least one processor that is configured to process data;
a transceiver that is configured to transmit data; and
a weight sensing system that is configured to measure the weight of at least one dietary item, wherein the weight of at least one dietary item is correlated to the caloric content of said dietary item.

19. A connected tableware system, comprising:
a connected tableware system having a holding surface that is configured to at least partially support at least one dietary item, a data processing unit that is configured to store and process data, a transceiver that is configured to transmit data, and a measurement sensor that is configured to measure at least one characteristic of at least one dietary item; and
a cloud computing network having at least one data processing unit that is configured to store and process data and a transceiver that is configured to receive and transmit data.

20. The connected tableware system of claim 19, further comprising a data transfer medium having a transceiver that is configured to receive and transmit data, a data processing unit that is configured to store and process data, and a display that is configured to show data.
